(19) United States
(12) Patent Application Publication
(10) Pub. No.: US 2018/0311438 A1
(43) Pub. Date: Nov. 1, 2018

(54) Device For Automatically Injecting Liquid Product Comprising An Injection Spring

(71) Applicant: Nemera La Verpillière S.A.S., LA VERPILLIERE (FR)

(72) Inventors: Kevin Stamp, Sheffield (GB); Pascal Dugand, Estrablin (FR)

(21) Appl. No.: 15/747,340

(22) PCT Filed: Jul. 22, 2016
(86) PCT No.: PCT/FR2016/051912

§ 371 Date: Jan. 24, 2018

(30) Foreign Application Priority Data
Jul. 24, 2015 (FR) 1557075

(51) Int. Cl. A61M 5/20; A61M 5/32

(52) U.S. Cl. CPC A61M 5/2033; A61M 5/3202; A61M 2205/0216; A61M 2005/2073; A61M 2005/2086

(57) ABSTRACT

An automatic injection device including an injection spring deformable between a compressed state prior to use and a relaxed state after injection. The device includes a plunger control member configured to apply a force to a plunger under the action of the injection spring to allow the plunger to move in the distal direction of the device. The plunger control member bears a stop that is retractable between an active configuration in which it retains the injection spring in the compressed state and a retracted position in which it releases the injection spring. The device also includes a locking member for locking the retractable stop in its active configuration, this locking member being able to adopt a locking position in which it applies pressure to the retractable stop to prevent it from adopting its retracted configuration, and an unlocking position in which the retractable stop is released.

### FIELD OF THE INVENTION

The present invention relates to the field of automatic injection devices for liquid products, more particularly in the pharmaceutical field.

### BACKGROUND OF THE INVENTION

An automatic injection device is generally a medical device allowing the automatic administration of a liquid medication requiring injection. In particular, these devices allow patients suffering for example from rheumatoid arthritis, multiple sclerosis, or diabetes or having suffered anaphylactic shock due to allergy to independently inject themselves with the dose of medication.

An example of an automatic injection device is described in the document U.S. Pat. No. 8,734,402. The device comprises an injection syringe that contains the product to be injected and is equipped with a needle and a syringe support. Brief pressure resulting from application to the skin of the patient is generally sufficient to cause penetration of the needle into the skin, followed by an injection of the liquid product and then retraction of the needle inside the device so as to avoid injuring a person with the needle.

More particularly, the automatic injection device comprises two springs under compression. A first spring is housed at the proximal end of the device and exerts a certain force capable of automatically displacing the elements, including the injection needle, in order to cause the injection needle to penetrate into the body of the patient and trigger injection of the liquid product to be injected. The function of the second spring is to cause the injection needle to be retracted inside the device after injection is completed.

It is important to prevent unintentional triggering of the springs before use of the automatic injection device, particularly during transportation or in the case of accidental dropping of the automatic injection device. Indeed, in the case of accidental release of one of the springs before use, the automatic injection device becomes unusable.

The document U.S. Pat. No. 6,203,530 describes an automatic injection device comprising retractable stops but not comprising members for locking these stops.

The present invention proposes an automatic injection device having improved reliability.

For this purpose, the object of the invention is to provide an automatic injection device for liquid products having a distal end and an opposite proximal end, comprising:
an injection spring that is deformable between a compressed state prior to use of the automatic injection device and a relaxed state after injection,
a plunger control member configured to apply a force to a plunger under the action of the injection spring in order to allow the plunger to move in the distal direction of the automatic injection device, the plunger control member carrying a stop that is retractable between an active configuration in which it retains the injection spring in the compressed state and a retracted configuration in which it releases the injection spring,
a locking member for locking the retractable stop in its active configuration, this locking member being able to assume a locking position in which it exerts pressure on the retractable stop to prevent it from assuming its retracted configuration and an unlocking position in which the retractable stop is released to assume its retracted configuration.

Therefore, by means of the locking member, it becomes impossible for the retractable stop to accidentally pass into the retracted configuration and thus release the injection spring. This locking is particularly useful during transportation or storage of the automatic injection device prior to its use. Moreover, locking is also highly advantageous in the case of accidental dropping of the automatic injection device, in particular immediately prior to its use. Moreover, this locking member is particularly important in the case of use of a relatively powerful injection spring, because it is more difficult in such cases to maintain the retractable stop in its active configuration because of the force of the spring. It therefore becomes possible to use particularly rigid springs and thus to inject a product having a higher viscosity than previously. In particular, it is possible to use an injection spring having a force in a compressed position of 20 N, even 50 N, or as high as 80 N or more.

### SUMMARY OF THE INVENTION

In the present description, the injection axis corresponds to the axis of the automatic injection device, which corresponds to the axis of the injection needle. The injection axis will be referred to as X. Moreover, the distal direction refers to the direction the farthest from the user's fingers, i.e. the closest to the skin of a patient at the time of injection, and the proximal direction refers to the opposite direction. In other words, one could say that the “distal direction” is the one towards the “front” of the automatic injection device. In particular, the distal end of a piece corresponds to the end located on the side of the injection needle, and the proximal end corresponds to the opposite end.

The automatic injection device can further comprise one or a plurality of the following claims, taken individually or in combination.

The locking member comprises a central locking pin provided in the proximal end of an outer housing of the automatic injection device. It is therefore particularly simple and economical to implement.

The proximal end of the outer housing comprises a radial surface, recessed in the vicinity of the unlocking pin so as to be crossed by an elastic tab carrying the retractable stop at least when the locking member is in the locking position.

The locking pin comprises two sections distributed axially and of different diameters, namely a locking section and an unlocking section, the locking section having a radial dimension that is greater than a radial dimension of the unlocking section when viewing an axial section through the retractable stop, such that the locking section exerts a radial pressure on the retractable stop when the locking member is in the locking position, and the unlocking section creates a space for disengaging the retractable stop when the locking member is in the unlocking position.

The locking member carries a ramp configured to cooperate by ramp effect with an elastic tab carrying the retractable stop in order to shift it from the active configuration to the retracted configuration.

The passage from the locking position to the unlocking position of the locking member is triggered by application of the distal end of the automatic injection device to the skin of a patient. In this manner, unlocking is not triggered until the moment before injection, without the user being aware of this. This is particularly advantageous in case the user drops the automatic injection device immediately before proceeding with injection.

The retractable stop is radially displaced when it is in its active configuration, in particular such that it assumes a radially projecting position, and the retractable stop is radially retracted when it is in its retracted configuration, in particular such that it assumes a radially retracted position.

The retractable stop is carried by an elastic tab extending axially towards the proximal end of the automatic injection device, the elastic tab being deformable between a radially displaced rest configuration giving the retractable stop its active configuration and a radially retracted configuration giving the retractable stop its retracted configuration.

The retractable stop in its active configuration rests against a complementary surface carried by a spring retaining ring, this retaining ring being attached to a position control member comprising a cam track in order to control the movement of elements of the automatic injection device under the action of the injection spring.

The plunger control member comprises two diametrically opposed semi-annular elastic tabs, each carrying a retractable stop, with each semi-annular elastic tab having a longitudinal window allowing deformation to facilitate the assembly of the injection spring and a spring retaining ring around the two semi-annular elastic tabs.

### BRIEF DESCRIPTION OF THE DRAWINGS

The invention will be better understood with reference to the attached figures, which are provided by way of example and are by no means limitative, in which:

FIG. 1 is a perspective view of an automatic injection device according to an embodiment,

FIG. 2 is an exploded perspective view of the automatic injection device of FIG. 1,

FIGS. 3 and 4 are perspective views respectively from the proximal end and the side of a member for withdrawing the protective cap of the automatic injection device of FIG. 1,

FIGS. 5 and 6 are perspective views respectively from the proximal end and the distal end of an end section of the automatic injection device of FIG. 1,

FIG. 7 is an exploded perspective view of the member for withdrawing the protective cap and the end section of the automatic injection device of FIG. 1,

FIGS. 8 and 9 are full and axial sectional perspective views respectively of a syringe support of the automatic injection device of FIG. 1,

FIG. 10 is an axial sectional perspective view of the end sleeve, syringe support, and protective springs of the automatic injection device of FIG. 1,

FIGS. 11, 12 and 15 are perspective side views respectively of a positioning rod, a plunger rod, and a needle control member of the automatic injection device of FIG. 1,

FIGS. 13 and 14 are transverse and axial sectional views respectively of the syringe support in which the injection syringe, the positioning rod, and the plunger rod of the automatic injection device of FIG. 1 are assembled,

FIGS. 16 through 18 and 20 are perspective side views respectively of a plunger control member, a retaining ring, a position control member, and an outer housing of the automatic injection device of FIG. 1,

FIG. 19 is a perspective sectional view from the distal end of the outer housing of FIG. 20,

FIG. 21 is a partial perspective view from the proximal end of the plunger control member, the plunger rod, and the retaining ring during the injection of liquid products of the automatic injection device of FIG. 1,

FIGS. 22 and 23 are partial axial sectional views of the automatic injection device of FIG. 1, showing a locking member in the locking and unlocking position respectively,

FIGS. 24, 25, 27 and 28 are axial sectional views of the withdrawal member, the end sleeve, the syringe support, and the injection syringe of the automatic injection device of FIG. 1, respectively at the time of insertion of the injection syringe into the syringe support; with the injection syringe inserted, in storage configuration; with the protective cap removed and before insertion of the injection needle into the skin of a patient; and with the injection needle inserted into the skin of a patient,

FIG. 26 is an axial sectional perspective view of the end sleeve, the syringe support, and the injection syringe of the automatic injection device of FIG. 1, showing the removal of the protective cap,

FIGS. 29 and 31 to 34 are perspective views and partial axial sectional views of several assembled elements of the automatic injection device of FIG. 1, respectively in storage position before removal of the protective cap; during the insertion of the injection needle into the skin of a patient; during the injection, with the plunger control member shown in full in FIG. 32 and in axial section in FIG. 33, and in protection position, and

FIG. 30 is an axial sectional perspective view of the automatic injection device of FIG. 1 shortly after pressing the distal end of the automatic injection device against the skin of a patient.

### DETAILED DESCRIPTION OF THE INVENTION

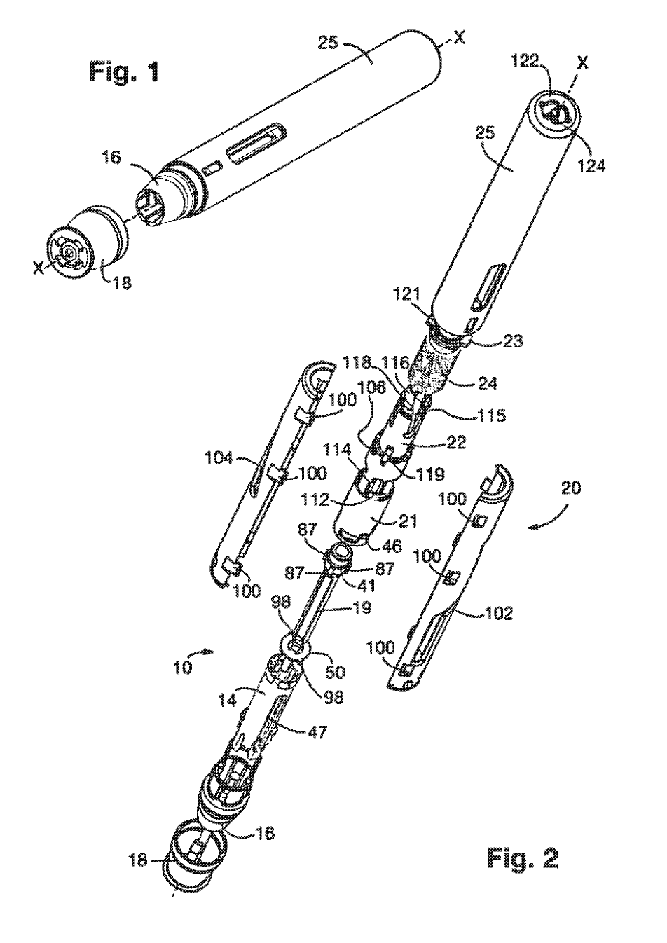

An automatic injection device for liquid products 10, as shown in FIG. 1, allows the automatic administration of a liquid product requiring injection, specifically a medicinal product. It is designed to receive an injection syringe 12 (shown in particular in FIG. 26) containing the liquid product. As shown in FIGS. 2 and 30, the automatic injection device 10 comprises a certain number of pieces, more particularly in this example a syringe support 14, two protective springs 15a, 15b, an end section 16, a member 18 for withdrawal of the protective cap 42, a plunger rod 19, a positioning rod 50, a position control member 20, a needle control member 21, a plunger control member 22, a retaining ring 23, an injection spring 24, and an outer housing 25.

As can be seen for example in FIG. 26 or FIG. 30, the injection syringe 12 comprises a syringe body 26 of a generally tubular shape around an injection axis X. The syringe body 26 comprises, at its distal end 26D, a distal shoulder 30 carrying an injection needle 32. The proximal end 26P of the syringe body 26 comprises a flange 38. A sliding plunger 40, of a generally cylindrical shape around the injection axis X, is mounted in the syringe body 26 and allows injection of a liquid product contained in the syringe body 26 via the injection needle 32. As shown in FIG. 14, the plunger 40 has a distal end disposed on the side of the liquid product and an opposite proximal end 40P. The injection needle 32 is covered by a protective cap 42 of the RNS (rigid needle shield) type comprising an internal stopper of soft rubber designed to receive the end of the injection needle 32 and ensure that it is sealed, with the internal stopper being enclosed by a shell of rigid plastic material that provides it with favorable mechanical strength. This protective cap 42 has a generally cylindrical shape around the injection axis X, and can have a diameter greater than that of the syringe body 26, which, at the end of the stroke, rests against the distal shoulder 30. In this example, the injection syringe 12 is a prefilled glass syringe with an attached needle having a volume of 1 mL (milliliter). It will be noted that the syringe body 26 delimits a maximum volume of liquid received, but that it is possible to fill it only partially by moving the plunger 40 towards the distal end 26D of the syringe body 26.

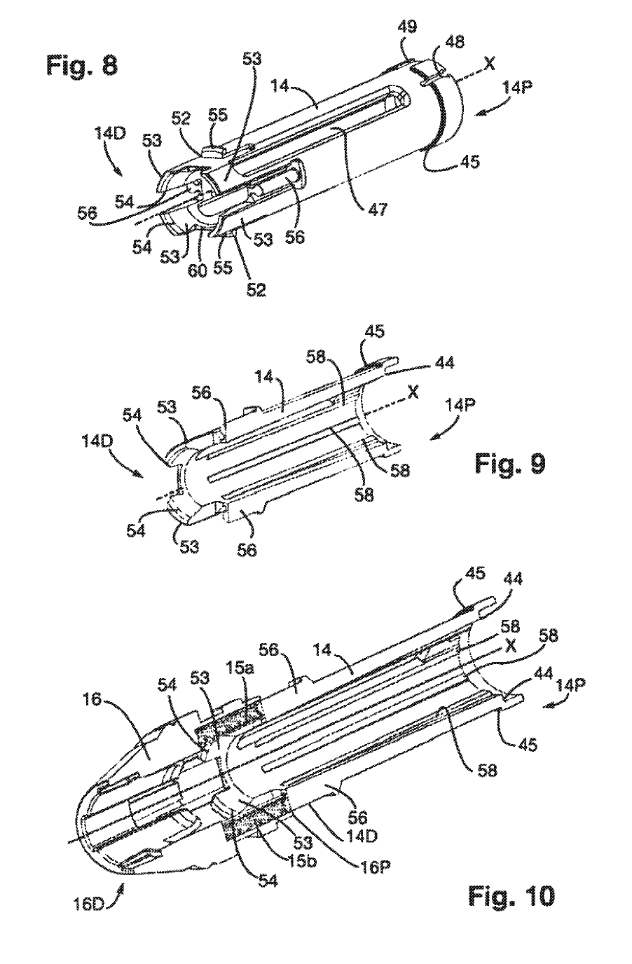

As shown in FIG. 8, the syringe support 14 is of a generally tubular shape around the injection axis X, it is open at its two ends, and it is designed to receive the injection syringe 12. The syringe support 14 is thus a distinct single piece composed of two half shells assembled together. The syringe support 14 is configured to house the injection syringe 12 such that it is mounted in a fixed position in the syringe support 14 throughout operation of the automatic injection device 10. The syringe support 14 has a distal end 14D disposed on the side of the injection needle 32 and an opposite proximal end 14P.

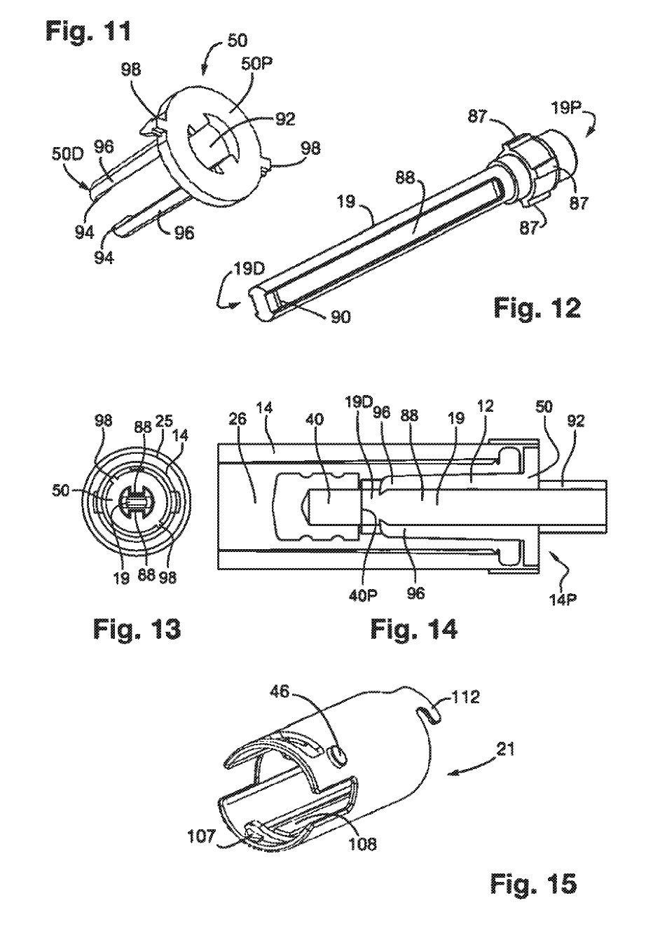

As can be seen in FIGS. 8 and 9, at its proximal end 14P, the syringe support 14 comprises an internal clearance 44 delimited by an internal shoulder, which allows it to receive the flange 38 of the syringe body 26 when the injection syringe 12 is inserted into the syringe support 26. This internal clearance 44 allows the proximal end 14P to have an internal diameter greater than the rest of the syringe support 14, and it can thus accommodate on the one hand the flange 38 and on the other a positioning ring 50 of the plunger rod 19, with these two elements having a diameter greater than the syringe body 26. The proximal end 14P of the syringe support 14 further comprises an external shoulder 45 which is complementary to the internal shoulder 44. This external shoulder 45 forms a blocking relief of the needle control member 21. More particularly, the external shoulder 45 allows the needle control member 21 to be hooked onto the proximal end 14P of the syringe support 14 during assembly of a proximal and distal assembly of the automatic injection device 10 and until completion of insertion of the injection needle 32 into the skin of the patient. Moreover, two axial notches 48 are provided opposite each other in the wall of the proximal end of the syringe support 14. These axial notches 48 allow cooperation with the positioning ring 50 of the plunger rod 19 shown in particular in FIGS. 11 and 13. The external shoulder 45 further comprises a recess 49 forming an axial guiding rail for a guide rib 108 of the needle control member 21.

Two viewing windows 47 are provided opposite each other in the wall of the syringe support 14. Each viewing window 47 is of an elongated shape, extends essentially in the direction of the injection axis X, and allows the injection syringe 12 to be observed when it is inserted into the syringe support 14. This further allows the user to see that there is liquid product in the injection syringe 12. Moreover, as shown in FIG. 9, the syringe support 14 comprises, on an internal surface of its wall capable of being in contact with the injection syringe 12, a damping element 58 made of an elastomer material, for example in the form of an elastomer band extending axially and also acting as an entering rib. A plurality of damping elements 58 is distributed angularly on the internal surface of the syringe support 14. They are designed on the one hand to center the injection syringe 12 when it is housed in the syringe support 14 and on the other to absorb a part of the pressure exerted on the injection syringe 12 on activation of the automatic injection device 10. Moreover, they contribute towards axial blocking of the injection syringe 12 in the syringe support 14, in particular when the injection syringe 12 is advanced in order to carry out insertion and during the pressure exerted by the plunger 40 on the injection syringe 12 during injection. Indeed, the friction between the elastomer material and the syringe body 26 makes it possible for the pressure received by the syringe body 26 to be directly transferred to the syringe support 14 in order to prevent breakage of the injection syringe 12, which is often made of glass. These damping elements 58 make it possible to absorb the impact received by the automatic injection device 10, for example during transportation, thus protecting the injection syringe 12 from possible risks of breakage. For example, the elastomer material can be a thermoplastic elastomer (TPE) material, preferably injection molded or over-molded.

As can be seen in FIG. 8, at its distal end 14D, the syringe support 14 has on either side of the viewing windows 47 means for retaining the end section 16 on the syringe support 14 comprising two radial projections 52 located on the external surface of the syringe support 14, essentially opposite each other. Each radial projection 52 comprises, on its distal surface, a ramp 55 designed to cooperate with the end section 16 during its assembly by snap-locking onto the syringe support 14. The distal end 14D of the syringe support 14 also comprises holding tabs 53, more particularly four holding tabs 53 extending axially, distributed angularly along the distal contour of the syringe support 14. Each holding tab 53 comprises a bevelled surface 54 designed to cooperate with the end section 16 when the latter is in the injection position. The distal end of the syringe support 14 also comprises guiding means of the end section 16 on the syringe support 14. These guiding means comprise a guide form in relief, namely two guide pins 56 that are integral with the syringe support 14 extending on either side of the external wall of the syringe support 16 along the injection axis X. Each of the guide pins 56 is designed to cooperate with a guide in a complementary recess, specifically a slide 57 (shown in FIG. 6), and is provided on the end section 16 comprising a passage slit in order to allow guiding during relative movement of the end section 16 and the syringe support 14. Each slide 57 has a distal part delimiting a housing for receiving one of the protective springs 15a or 15b. In this manner, once they are released, each of the protective springs 15a, 15b can exert a force on one of the guide pins 56 in order to allow relative sliding of the end section 16 and of the syringe support 14, with the protective springs 15a, 15b being in compression. As can be seen in FIGS. 9 and 10, the distal end 14D of the syringe support 14 further comprises two notches 60 allowing the passage of hooking tabs 66 provided on the withdrawal member 18 of the protective cap 42. The notches 60 are axially aligned with the retaining means 52.

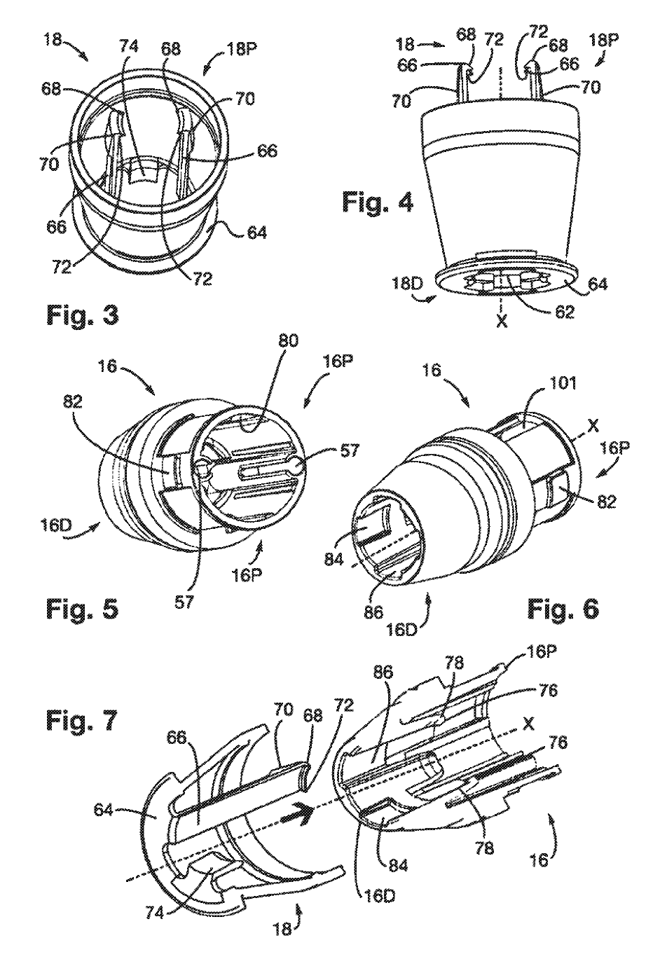

The withdrawal member 18 of the protective cap 42, shown in FIGS. 3, 4, and 7, forms the distal end of the automatic injection device 10. It is designed to be withdrawn from the automatic injection device 10 before activation thereof. The withdrawal member 18 has a general frustoconical shape around the injection axis X, open at the two ends, with a proximal diameter greater than the distal diameter. The distal end of the withdrawal member 18 further comprises, around its distal opening 62, a gripping means for a user comprising a tapered base 64 and further allowing the automatic injection device 10 to stand upright when it is placed on a horizontal support. The withdrawal member 18 comprises two hooking tabs 66 of the protective cap 42. The two hooking tabs 66 are located inside the withdrawal member 18 and are diametrically distributed around the injection syringe 12 when the withdrawal member 18 is mounted on the automatic injection device 10. The two hooking tabs 66 extend axially (with respect to X) from the distal opening 62 towards the proximal end of the withdrawal member 18 and project from this proximal end.

As can be seen in FIG. 4, each hooking tab 66 comprises a proximal ramp 68 cooperating with the lateral external wall of the protective cap 42 on insertion of the injection syringe 12 into the syringe support 14, as well as an external ramp 70 cooperating with the end section 16 on which the withdrawal member 18 is mounted. Each hooking tab 66 further comprises a shoulder 72 designed to cooperate with the proximal edge of the protective cap 42 of the injection syringe 12 and allowing its withdrawal when a user pulls axially on the withdrawal member 18. In addition, each external ramp 70 makes it possible, when a user pulls axially on the withdrawal member 18, by cooperation with the end section 16, to tighten the hooking tab 66 against the protective cap 42. In this manner, the withdrawal member 18 is capable of carrying along the protective cap 42 when it is withdrawn from the automatic injection member 10. The withdrawal member 18 is removably fixed on the distal end of the end section 16, and thus itself forms a means for gripping the protective cap 42 (as shown in FIG. 26). The withdrawal member 18 further comprises, on either side of the hooking tabs 66, on the internal surface of its wall, two lugs 74 cooperating with the end section 16.

As shown in FIGS. 1 and 2, the end section 16, after withdrawal of the withdrawal member 18, forms the distal end of the automatic injection device 10. It is thus designed to be in contact with the skin of a patient during the injection. The end section 16 is moved back onto the distal end 14D of the syringe support 14. It is slidably mounted with respect to the syringe support 14 between an injection position, in which the syringe support 14 and the end section 16 are axially (with respect to X) close together, and a protection position in which the syringe support 14 is axially moved away, in the distal direction, from the end section 16. The sliding of the end section 16 with respect to the syringe support 14 is made possible by the cooperation of the rails 57 provided in the internal wall of the end section 16 and the guide pins 56 carried by the external wall of the syringe support 14, forming means of cooperation between the end section 16 and the syringe support 14. The end section 16 has a general frustoconical shape around the injection axis X, open at the two ends, with the proximal diameter being greater than the distal diameter.

As can be seen in FIGS. 26 and 28, the end section 16 comprises two retaining stops 76, each being carried by an elastic tab 78. Each elastic tab 78 extends axially (with respect to X) from the distal end towards the proximal end of the end section 16 and projects from the internal surface of the end section 16. The two elastic tabs 78 are symmetrical to each other with respect to an axis of symmetry passing through the injection axis X. In this manner, each elastic tab 78 extends axially (with respect to X) and has a proximal end having a surface extending along an essentially radial plane, this surface forming an axial retaining stop 76 of the injection syringe 12, preventing the injection syringe 12 from unintentionally moving towards the distal end of the automatic injection device 10. Each elastic tab 78 is deformable between a tightened position, configured to axially (with respect to X) retain in the distal direction the injection syringe 12 in the syringe support 14, and a spaced position configured to allow axial (with respect to X) insertion in the distal direction, from the proximal end of the syringe support 14, of the injection syringe 12 equipped with the protective cap 42. When the end section 16 is in injection position, as seen in FIG. 28, the holding tabs 53 of the syringe support 14 maintain the elastic tabs 78 in a tightened position, the holding tabs 53 thus preventing the elastic tabs 78 from assuming their spaced position and thus preventing the injection syringe 12 from moving back (toward the proximal end of the automatic injection device 10). Moreover, when the end section 16 is in injection position, it encloses the holding tabs 53 so as to prevent moving apart of the holding tabs 53 resulting from moving apart of the elastic tabs 78.

As can be seen in FIGS. 5 and 6, the end section 16 further comprises, at its proximal end, means for retaining the end section 16 on the syringe support 14, comprising two proximal windows 80 designed to serve as guides for sliding of the two radial projections 52 of the syringe support 14, which are assembled in the proximal windows 80 by snap-locking. The proximal end of the end section 16 further comprises, on its lateral external wall, two projecting lugs 82 (cf. FIGS. 5 and 6) radially configured to cooperate with the position control member 20 in order to keep it in place.

In order to ensure that the withdrawal member 18 does not unintentionally become detached from the end section 16, the end section 16 further comprises, on the distal part of its internal wall, means for retaining the withdrawal member 18 comprising two recesses 84 designed to cooperate by clamping or snap-locking with the two lugs 74 of the withdrawal member 18 (cf. FIGS. 6 and 7). This thus prevents the rotation of the protective cap 42 with respect to the end section 16, rotation which could cause the cutting edge of the bevel of the injection needle 32 to cut out a piece of the internal stopper of the protective cap 42, a phenomenon known as “coring”. In order to allow the withdrawal of the withdrawal member 18 on the end section 16 after the insertion of the injection syringe 12 into the syringe support 14, the end section 16 also comprises, on the distal part of its internal wall, on either side of the two recesses 84, guiding means of the withdrawal member 18 comprising two axial rails (with respect to X) 86. It will be understood that the end section 16 is brought back around the distal end of the syringe support 14, such that the syringe support 14 slides inside the end section 16.

The plunger rod 19 makes it possible to transfer pressure onto the plunger 40 in order to inject the liquid product. It comprises, as can be seen in FIG. 12, a distal end 19D facing towards the plunger 40 and designed to be in contact with the proximal end 40D of the plunger 40, during the injection in particular. This distal end 19D of the plunger rod 19 has a cross-section with a foolproof shape, more particularly a non-circular shape. The plunger rod 19 further comprises a proximal end 19P opposite the distal end 19D configured to cooperate with the plunger control member 22 activated by the injection spring 24. The proximal end 19P of the plunger rod 19 is of an overall cylindrical shape around the injection axis X and comprises four radial projections 87, distributed angularly around the injection axis X, designed to cooperate with the plunger control member 22. As can be seen in FIG. 12, two sliding grooves 88 are provided in the plunger support 19, essentially opposite each other and diametrically opposed. Each sliding groove 88 can, for example, be formed by an axial slot. These two sliding grooves 88 extend axially (with respect to X) along the plunger rod 19 and delimit the foolproof shape of the cross-section. These sliding grooves 88 are designed to cooperate with an alignment cam of the positioning rod 50. The plunger rod 19 further comprises, provided on its distal end 19D, a distal stop 90 for axial retention of the positioning rod 50, more particularly two diametrically opposed distal stops. Preferably, the distal stop 90 is formed by a transverse slot provided in each of the sliding grooves 88.

The positioning rod 50, shown in FIG. 11, is designed to cooperate with the plunger rod 19 in order both to allow favorable axial and angular positioning of the plunger rod 19 and to prevent withdrawal of the plunger rod 19 outside the operating ring 50 and even the automatic injection device 10. The axial positioning makes it possible ensure a correct position with respect to the plunger 40 on the one hand and the plunger control member 22 on the other. The angular positioning not only makes it possible to prevent the plunger rod 19 from rotating on itself during activation of the plunger rod 19 but also ensures a preset angular orientation with respect to the syringe support 14, allowing axial stopping and axial releasing of the plunger rod 19 and the plunger control member 22, these configurations being implemented following a rotation of the plunger control member 22 with respect to the plunger rod 19. The positioning rod 50 is designed to be brought back onto the proximal end 14P of the syringe support 14. In order to prevent free rotation of the positioning rod 50 with respect to the syringe support 14, the positioning rod 50 comprises angular clamping means 98 of the positioning rod 50 on the proximal end 14P of the syringe support 14. These angular clamping means 98 can comprise, for example, external clamping lugs designed to be inserted into the axial notches 48 of the syringe support 14, as can be seen in FIG. 13 or FIG. 26. The axial positioning means of the plunger rod 19 provided on the positioning rod 50 comprise an axial retaining stop 94 of the plunger rod 19, carried here by an elastic tab 96 extending axially from a proximal washer 50P towards a distal end 50D of the positioning rod 50. More particularly, the positioning rod 50 comprises two diametrically opposed elastic tabs 96, each carrying an axial retaining stop 94, which is formed in this example by an axial retaining lug of the plunger rod 19. Each axial retaining stop 94, by cooperating with a corresponding distal stop 90 of the plunger rod 19, prevents full withdrawal (by traction in the proximal direction) of the positioning rod 50 before assembly and the injection syringe 12 after assembly. Moreover, the axial positioning means of the plunger rod 19 on the positioning rod 50 are disposed such that, in the storage configuration of the automatic injection device 10, there is clearance between the distal end 19D of the plunger rod 19 and the proximal end 40P of the plunger 40, preferably a clearance of between 0.5 and 2 mm, for example close to 1 mm, as can be seen in FIG. 14. The positioning rod 50 further comprises angular positioning means of the plunger rod 19 on the positioning rod 50 comprising a through opening 92 of the plunger rod 19 having a foolproof shape complementary to that of the distal end 19D of the plunger rod 19. In this manner, the through opening 92 ensures precise and controlled angular positioning of the plunger rod 19 with respect to the positioning rod 50 throughout operation of the automatic injection device 10.

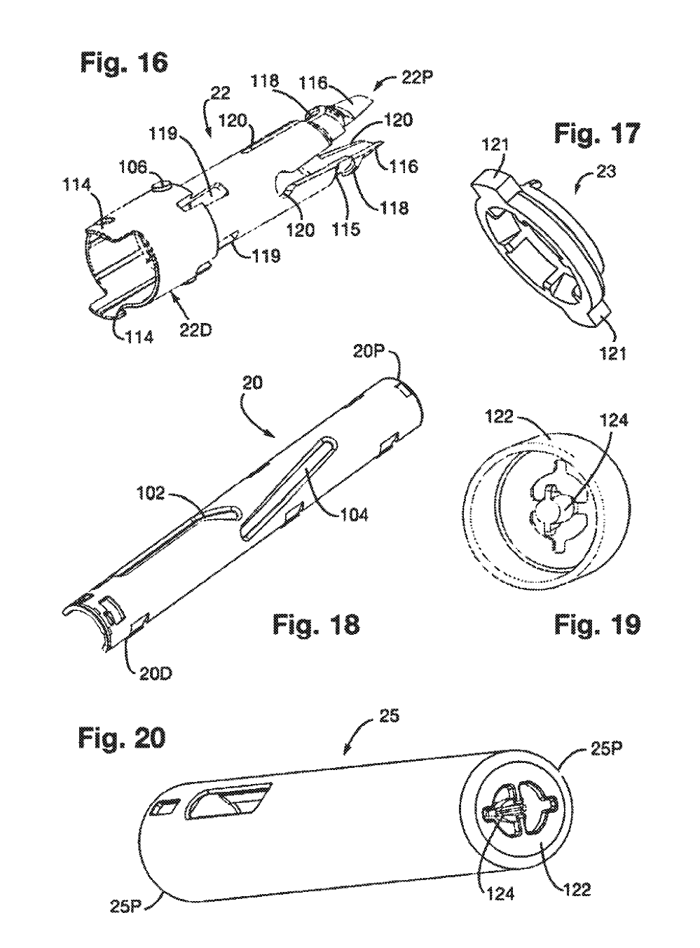

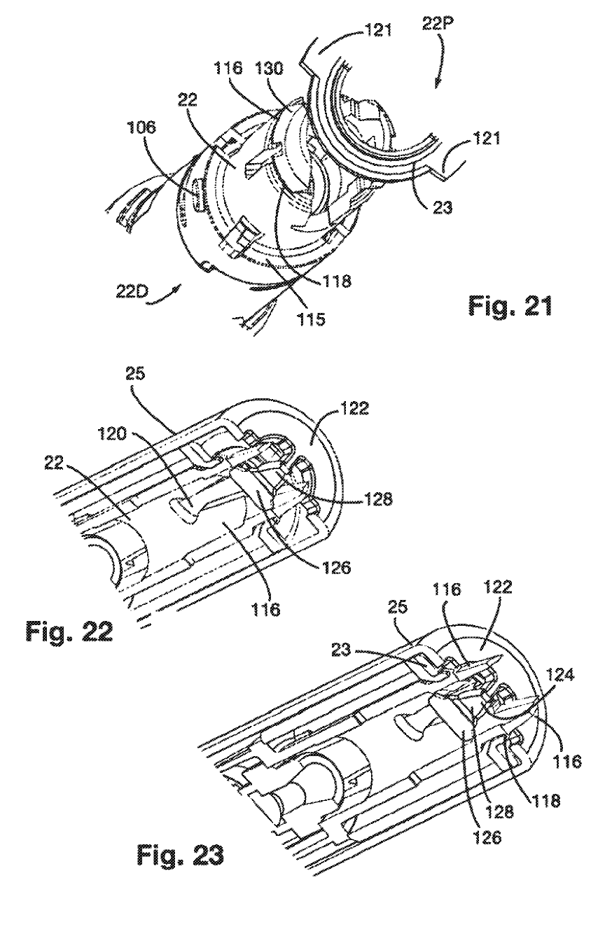

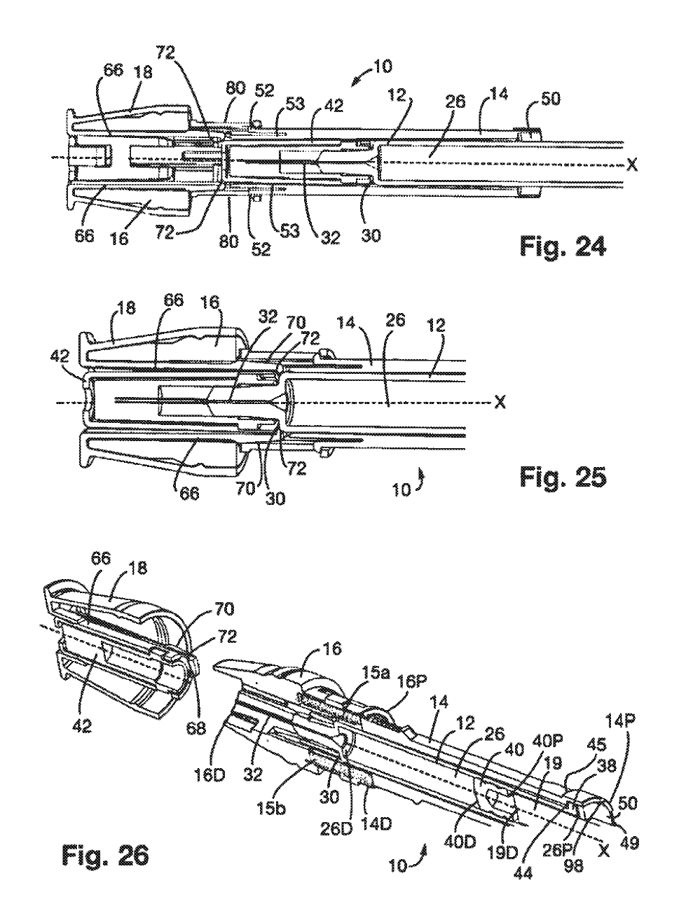

FIG. 2 shows the position control member 20, which comprises two parts, two half shells designed to be snap-locked into each other around the syringe support 14 by snap-locking means 100 comprising various notches and lugs. The position control member 20 thus has a generally tubular shape of axis X. The distal end of the position control member 20 is provided with retaining means of the end section 16 comprising a blocking window 101 designed to cooperate with the two projecting lugs 82 of the lateral external wall of the end section 16 (cf. FIG. 6). This blocking window 101 has a bayonet shape in order to allow the assembly of the end section 16 and the position control member 20 by means of a slight rotation. Once assembled, the end section 16 and the position control member 20 are fixed with respect to each other throughout operation of the automatic injection device 10. As can be seen in FIG. 18, the position control member 20 also comprises first and second cam tracks 102 and 104 designed to cooperate respectively with first and second cams 46, 106 for position control carried respectively by the needle control member 21 (FIG. 15) and the plunger control member 22 (FIG. 16). In this manner, as the needle control member 21 and the plunger control member 22 have different cam tracks, once the automatic injection device 10 is activated, the step of insertion of the injection needle 32 into the body of the patient and the step of injection of the liquid product contained in the injection syringe 12 are different and separate steps so that each can be implemented in a precise, time-controlled fashion and a manner adapted to the liquid product injected. This also makes it possible to ensure that the injection needle 32 is inserted the proper distance into the skin of the patient 11 before the injection begins. Moreover, as can be seen in FIG. 18, the first cam track 102 has a part having a greater slope than the rest of the first cam track 102, specifically a slope of approximately 45°, which allows the injection needle 32 to be progressively advanced at the outset, thus limiting pain in the patient. The rate of advancement of the injection needle 32 can be controlled by changing the slope of the first cam track 102.

The needle control member 21 is a generally tubular shape, as can be seen in FIG. 15. At its proximal end 21P, it comprises provisional coupling means 112 with complementary coupling means 114 carried by the plunger control member 22 (FIG. 16), such that the needle control member 21 and the plunger control member 22 move integrally at the beginning of the push exerted by the injection spring 24. In this manner, when the injection spring 24 begins pushing on the plunger control member 22, it also pushes the needle control member 21 which, at this stage, is also coupled to the syringe support 14, and pushes the syringe support 14 in the distal direction, thus allowing the insertion of the injection needle 32 into the skin of the patient 11. More particularly, the provisional and complementary coupling means 112, 114 comprise two complementary parts with a mutually hooking shape, for example two inverted hooks extending axially, the first carried by the proximal end 21P of the needle control member 21 and the second carried by the distal end 22D of the plunger control member 22. Moreover, the needle control member 21 comprises an axial guide rib 108 that projects from the internal surface of the needle control member 21, and at its distal end 21D, a blocking pin 107 that projects from its internal surface and has a funnel shape, with the narrow part of the funnel facing the distal side. The blocking pin 107 is designed to cooperate with the external shoulder 45 of the proximal end 14P of the syringe support 14. Indeed, this external shoulder 45 forms a blocking relief of the needle control member 21 at the beginning of operation of the automatic injection device 10 during insertion of the injection needle 32 into the skin of the patient. As explained above, the external shoulder 45 thus makes it possible to hook the needle control member 21 onto the proximal end 14P of the syringe support 14 during assembly of the proximal and distal assemblies of the automatic injection device 10, with this assembly taking place by axial insertion of the blocking pin 107 into the guiding rail 49 of the syringe support 14, followed by rotation of the needle control member 21 with respect to the syringe support 14, so that when the automatic injection device 10 is in storage position prior to use, the external shoulder 45 is wedged axially (according to the injection axis X) between the blocking pin 107 of the distal side and the distal end of the guide rib 108 of the proximal side. The needle control member 21 further comprises, on its external surface and its distal end 21 D, the first cam 46, designed to cooperate with the first cam track 102 provided in the position control member 20.

The plunger control member 22, as can be seen in particular in FIG. 16, has a generally tubular shape. It is configured to apply a force on the plunger 40 (via the plunger rod 19) under the action of the injection spring 24. In other words, the plunger control member 22 is designed to transfer the action of the injection spring 24 to the plunger 40, more particularly via the plunger rod 19. This force allows axial movement (with respect to X) of the plunger 40 in the distal direction of the automatic injection device 10 in order to inject the liquid product into the skin of the patient. The plunger control member 22 carries a stop 118 that is retractable between an active retaining configuration of the injection spring 24 in the compressed state and a retracted configuration in which it releases the injection spring 24. More particularly, the control member 22 comprises two diametrically opposed semi-annular elastic tabs 116, which are semi-flexible, each carrying a stop that is retractable 118, and are designed to cooperate with the locking member of the outer housing 25. Each of the semi-annular elastic tabs 116 carrying the retractable stops 118 extends axially (with respect to X) towards the proximal end of the automatic injection device 10, each semi-annular elastic tab 116 therefore being deformable between a radially displaced rest configuration giving the retractable stop 118 its active configuration and a radially retracted configuration giving the retractable stop 118 its retracted configuration. Each semi-annular elastic tab 116 of the plunger control member 22 also has a longitudinal window 120 allowing deformation to facilitate the assembly of the injection spring 24 and the retaining ring 23 of the injection spring 24 around the two semi-annular elastic tabs 116. In this manner, each retractable stop 118 is radially displaced when it is in its active configuration. In particular, this allows each retractable stop 118 of the plunger control member 22 to assume a radially projecting position. Moreover, each retractable stop 118 is radially retracted when it is in its retracted configuration, in particular such that it assumes a radially retracted position. At its distal end 22D, the plunger control member 22 has a generally cylindrical shape carrying the complementary coupling means 114 and the second cam 106 and delimiting an external proximal shoulder 115, forming a seat for the injection spring 24. This proximal shoulder 115 is also penetrated by the openings, which extend towards the proximal end 22P so as to form axial slits 119. The plunger control member 22 is configured so as to be capable of moving in rotation with respect to the plunger rod 19 between a pushing configuration, during which its rests against the radial projection 87 and pushes against the plunger rod 19, and a configuration in which the plunger rod 19 is released, in which it does not rest against the radial projection 87 and frees up a passage, formed by the axial slits 119, so that the plunger rod 19 can be withdrawn in the proximal direction.

The retaining ring 23 allows the injection spring 24 to be retained in the compressed state. More particularly, before the injection, the injection spring 24 is maintained in its compressed state by means of the retractable stop 118 carried by the control member of the plunger 22, cooperating with a complementary surface carried by the retaining ring 23. In other words, the retractable stop 118 in its active configuration rests against a complementary surface carried by a retaining ring 23 of the injection spring 24. This retaining ring 23 is moved back onto the proximal end 20P of the position control member 20. For this purpose, it has two lugs 121, external and diametrically opposed, for clamping of the retaining ring 23 designed to cooperate with complementary notches of the position control member 20. Moreover, the distal end of the retaining ring 23 forms a proximal seat for the injection spring 24, and the proximal end forms a complementary stop surface for the retractable stop 118.

The injection spring 24 is the main engine of injection. It must therefore exert force sufficient on the one hand to cause the injection needle 32 to penetrate into the body of the patient and on the other to cause the plunger 40 to be displaced in the syringe body 26 in order to inject the liquid product. Its force is preferably greater than 20 N, even 50 N, or as great as 80 N for a relatively viscous liquid product or for a high syringe section/needle ratio. This injection spring 24 is deformable between a compressed state prior to use of the automatic injection device 10 and a relaxed state after injection of the liquid product contained in the syringe body 26. As explained above, before the injection, the injection spring 24 is maintained in its compressed state by means of the retractable stop 118 carried by the control member of the plunger 22, cooperating with a complementary surface carried by the retaining ring 23. This retractable stop 27 is in fact retractable between an active configuration for retaining the injection spring 24 in the compressed state corresponding to a radially projecting position for retaining the injection spring 24 and a retracted configuration in which it releases the injection spring 24, allowing it to relax.

The outer housing 25 is the piece which, in this example, encloses most of the pieces of the automatic injection device 10. In particular, it allows these pieces of the automatic injection device 10 to be isolated from the exterior. The outer housing 25 also comprises a locking member 122 of the retractable stop 118 in its active configuration, provided in the proximal end of an outer housing 25 of the automatic injection device 10. This locking member 122 can assume a locking position in which it exerts pressure on the retractable stop 118 to prevent it from assuming its retracted configuration, and an unlocking position in which the retractable stop 118 is released to assume its retracted configuration. More particularly, as can be seen in FIG. 20, the proximal end 25P of the outer housing 25 comprises a radial surface, recessed (in two separate openings facing each other) in the vicinity of an unlocking pin 124 so as to be passed through by the semi-annular elastic tabs 116 of the plunger control member 22 carrying the retractable stops 118, at least when the locking member 122 is in the locking position. The locking pin 122 comprises two sections distributed axially (with respect to X) and of different diameters, namely a locking section 126 and an unlocking section 128. The locking section 126 has a radial dimension that is greater than a radial dimension of the unlocking section 128 when viewing an axial section through the retractable stops 118, such that the locking section 126 exerts a radial pressure on the retractable stops 118 when the locking member 122 is in the locking position. As for the unlocking section 128, it creates a space for disengaging of the retractable stops 118 when the locking member 122 is in the unlocking position. The locking section 126 and the unlocking section 128 are distally offset with respect to each other. Moreover, the locking member 122 has a ramp 130 configured to cooperate by ramp effect with the semi-annular elastic tabs 116 of the plunger control member 22 carrying the retractable stops 118 in order to cause them to shift from their active configuration to their retracted configuration. In this manner, the unlocking section 128 is distally at least partly opposite to the ramp 130. As explained below, the passage from the locking position to the unlocking position of the locking member 122 is triggered by applying the distal end of the automatic injection device 10 to the skin of a patient.

The main operating steps of the automatic injection device 10 are described below.

As can be seen in FIG. 1, the automatic injection device 10 is presented to the user in the form of a tube a dozen centimeters in length. Only the outer housing 25 and the withdrawal member 18, or the end section 16 if the withdrawal member 18 is withdrawn, are visible and accessible to the user.

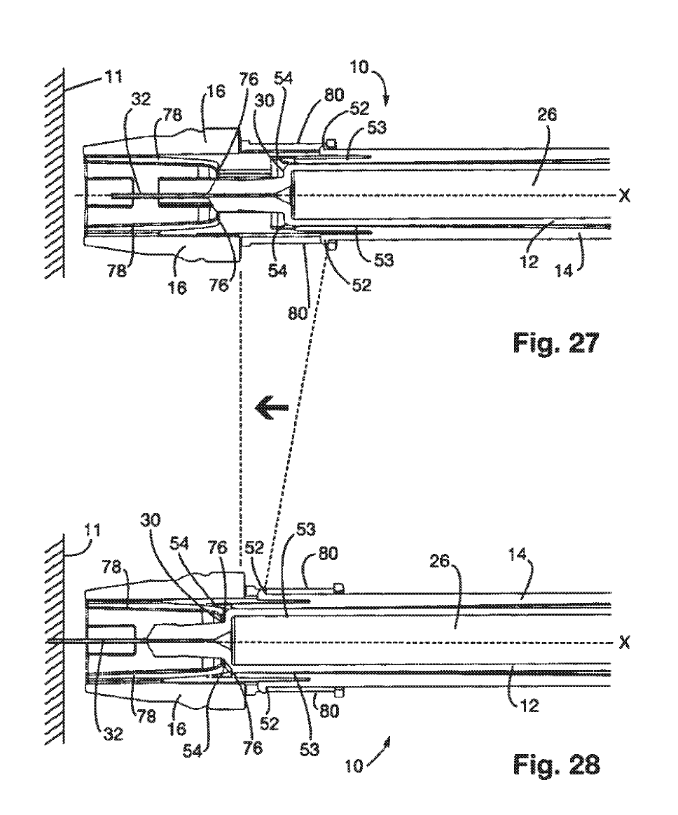

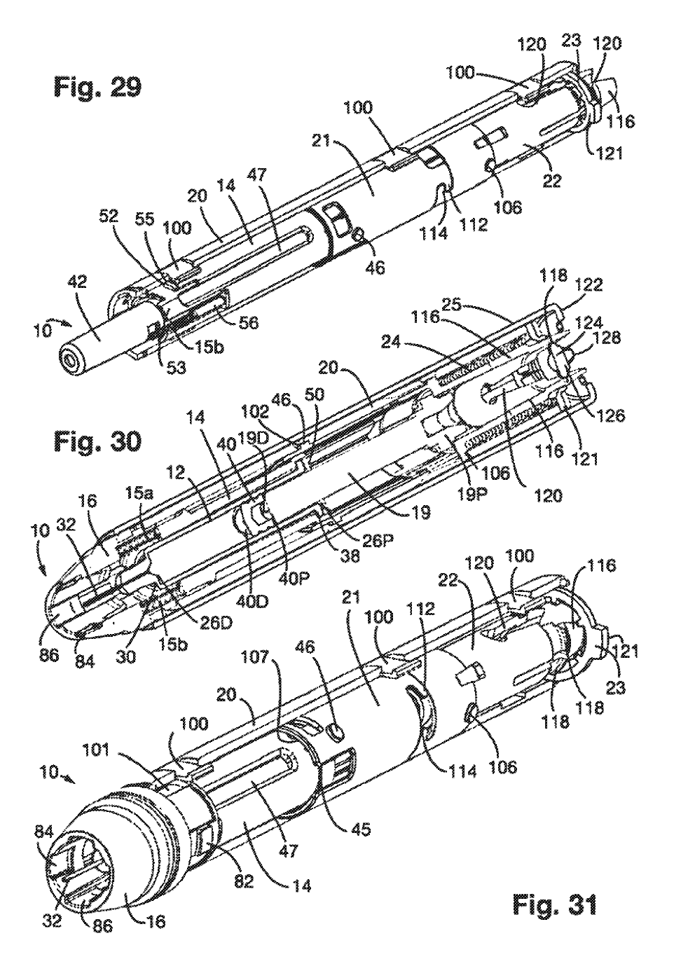

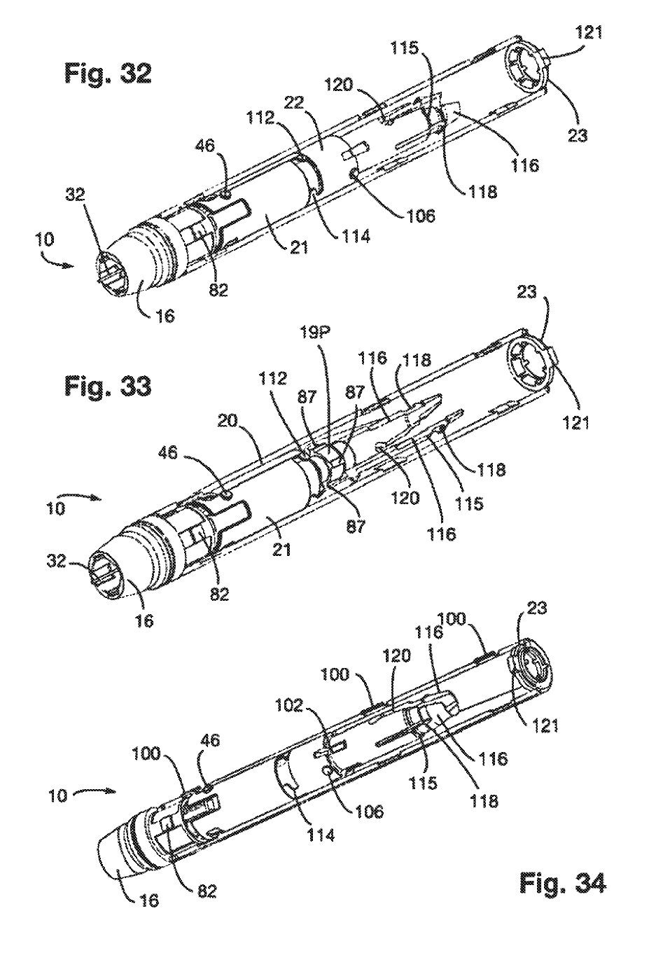

A first operating step consists of holding the withdrawal member 18 of the protective cap 42 in one hand and the outer housing 25 in the other hand and pulling on the withdrawal member 18 along the injection axis X in a distal direction. The axial traction movement activates the external ramp 70 of each hooking tab 66, tightening the two hooking tabs 66 against the protective cap 42, thus facilitating contact between the shoulder 72 of each hooking tab 66 and the proximal edge of the protective cap 42. In this manner, as shown in FIG. 15, the withdrawal of the withdrawal member 18 allows the withdrawal of the protective cap 42, thus exposing the injection needle 32. The injection needle 32 nevertheless remains inaccessible, because as shown in FIG. 17, it is enclosed by the end section 16. Once the withdrawal member 18 is withdrawn from the automatic injection device 10, the end section 16 forms the distal end thereof and the automatic injection device 10 is ready for use. At this stage, the injection needle 32 is withdrawn into the end section 16 by 3 mm (millimeters) in order to prevent accidental puncturing of the skin. According to a following step, the distal end of the end section 16 is placed against the skin of the patient 11 at the site at which injection of the medicinal liquid contained in the injection syringe 12 is desired. This step is followed by an unlocking step during which light axial pressure is applied in the distal direction to the outer housing 25. This pressure of the distal end of the automatic injection device 10 on the skin of the patient 11 triggers an axial movement of the outer housing 25 towards the end section 16. In other words, the end section 16 slides slightly inside the outer housing 25 towards its proximal end, producing sliding inside the outer housing 25 and towards the proximal end of the outer housing 25 of the position control member 20 and the retaining stop 23. This sliding results in unlocking of the retractable stop 118 carried by the proximal end of the plunger control member 22. The automatic injection device 10 is therefore located in its position prior to injection as shown in FIG. 27. The unlocking step is followed by a step of triggering automatic injection during which the user continues to exert axial pressure on the outer housing 25, pressing against the skin of the patient 11, in continuation of the axial pressure initiated in the unlocking step. This additional pressure produces a compression of the protective springs 15a, 15b which first allows the end section 16 and the syringe support 14 to approach each other axially (with respect to X). In this manner, the end section 16 slides further inside the outer housing 25 towards its proximal end, producing additional sliding, inside the outer housing 25 and towards the proximal end of the outer housing 25, of the position control member 20 and the plunger control member 22. Because of this, the retractable stop 118 carried by the plunger control member 22 slides towards the proximal end of the outer housing 25, which causes it to be retracted outside the grip of the retaining ring 23 by means of the ramp effect between the retractable stop 118 and the proximal surface of the outer housing 25, such that the injection spring 24 is released.

The release of the injection spring 24 triggers a series of reactions that end in penetration of the injection needle 32 into the body of the patient and injection of the liquid product contained in the syringe body 26. It will be noted that the stroke length of the injection needle 32 during these operations is 9 mm, such that the injection needle 32 protrudes by approximately 6 mm, corresponding to the depth of injection into the skin of the patient 11. More particularly, the injection spring 24 pushes in the distal direction on the plunger control member 22, which at this stage is coupled to the needle control member 21 due to meshing of the provisional and complementary coupling means 112, 114. The movement of the needle control member 21, which is integral with the syringe support 14 via the external shoulder 45, produces the movement in the distal direction of the syringe support 14. As the injection syringe 12 is mounted integrally with the syringe support 14, the injection syringe 12 is also pushed towards the distal end of the automatic injection device 10, and the injection needle 32 projects out of the end section 16. This axial convergence of the end section 16 and the syringe support 14 takes place by sliding on the one hand of the radial projections 52 of the syringe support 14 in the proximal windows 80 of the end section 16 and on the other of the guide pins 56 in the gutters 57 of the end section 16. This axial convergence is shown in FIG. 27 (before insertion of the injection needle 32) and FIG. 28 (after insertion of the injection needle 32). Once the injection needle 12 is inserted into the skin of the patient 11, the injection can begin. It will be noted that simultaneously with penetration of the injection needle 32, the first cam 46 of the needle control member 21 is guided by the corresponding first cam track 102 of the position control member 20, more particularly the part inclined to 45° of this first cam track 102. The result is that the speed of penetration of the injection needle 32 into the skin of the patient is lower than the unrestrained release speed of the injection spring 24, such that the pain associated with penetration of the injection needle 12 is advantageously reduced.

Simultaneously with the axial movement of the needle control member 21, a rotation of the plunger control member 22 with respect to the needle control member 21 is produced around the injection axis X by means of the second cam 106, which cooperates in the inclined second cam track 104. This rotation results in uncoupling of the needle control member 21 and the plunger control member 22, as well as coupling of the plunger control member 22 and the plunger rod 19, carried out by stopping of the control member of the plunger 22 against the proximal end 19D of the plunger rod 19, more particularly the radial projections 87. In this manner, the pressure exerted by the injection spring 24 on the plunger control member 22 results in pressure on the plunger rod 19, and thus on the plunger 40 in the syringe body 26, resulting in injection of the liquid product into the body of the patient.

It will be understood that the relative axial advance of the needle control member 21 and the plunger control member 22 is controlled by the cooperation of the first and second cams 46, 106 with the corresponding first and second cam tracks 102, 104 of the position control member 20. In this manner, the cooperation of the first and second cams 46, 106 of the needle control member 21 and the plunger control member 22 with the corresponding first and second cam tracks 102, 104 of the position control member 20 makes it possible to independently control the penetration of the injection needle 32 into the skin of the patient 11 and the injection of the medicinal liquid contained in the syringe body 26. It will be noted that the successive coupling mechanism, comprising coupling in a first step of the injecting needle control member 21 and the plunger control member 22 and coupling in a second step of the plunger control member 22 and the plunger rod 19, makes it possible to make penetration of the injection needle 32 and injection completely independent, i.e. one must wait until the injection needle 32 has penetrated to the proper depth before beginning injection, which prevents improper injection.

Once the injection is complete, the syringe support 14 returns freely into the plunger control member 22 because of rotation of the latter, which makes it possible to align the radial projections 87 of the plunger rod 19 with the axial slits 119, thus freeing a passage so that the plunger rod 19 can be withdrawn with respect to the plunger control member 22 in the proximal direction. The syringe support 14 is thus axially distanced from the end section 16, and the injection needle 32 is retracted into the end section 16, thus preventing any risk of injury. The two protective springs 15a, 15b can freely relax, they are no longer subjected to the force exerted by the injection spring 24, and they create a protective position in which the syringe support 14 is axially distanced from the end section 16, resulting in retraction of the injection needle 32 into the end section 16 and thus preventing any risk of injury.

It will be noted that throughout the duration of the injection in particular, the injection syringe 12 is placed under pressure, and it is retained in the automatic injection device 10, while the pressure exerted on the proximal end of the plunger rod 19, and thus on the plunger 40, can be considerable. As can be seen in FIG. 28, throughout the duration of the injection, it is the cooperation between the distal shoulder 30 of the injection syringe 12 and the two retaining stops 76 carried by the elastic tabs 78 of the end section 16 that makes it possible to retain the injection syringe 12, i.e. it is the distal end of the injection syringe 12 that is subject to the constraint resulting from the injection spring 24 applied to the plunger 40. In other words, while the injection spring 24 exerts pressure on the plunger rod 19 in order to displace the plunger 40 in the syringe body 26, the elastic tab 78 of the end section 16 ensures the axial retention (with respect to X) of the injection syringe 12 in the distal direction of the automatic injection device 10 such that the pressure exerted by the injection spring 24 on the plunger rod 19 during injection generates pressure on the distal end of the injection syringe 12, but does not generate pressure at the level of the flange 38 of the injection syringe 12. As the flange of an injection syringe is a fragile component, the risks of breakage of the injection syringe 12 are thus considerably reduced, and the injection spring 24 can be selected from among highly rigid springs, allowing for example the injection of products of high viscosity or up to 100 CP and/or the use of injection needles 32 having a reduced diameter.

According to a variant embodiment not shown, the flange can rest on an elastic ring, allowing most of the axial pressure to be taken up by the distal end of the injection syringe, thus reducing the pressure exerted on the body of the syringe at the level of the flange.

Moreover, it will be noted that throughout the duration of the injection, the end section 16 encloses the four holding tabs 53 of the syringe support 14 so as to prevent the holding tabs 53 from being separated as a result of moving away of the elastic tab 78 under the effect of the pressure exerted by the distal shoulder 30 of the injection syringe 12 on the retaining stops 76. In this manner, the holding tabs 53 are maintained in their tightened position by the proximal part of the end section 16. By means of the stop effect between the bevelled surfaces 54 and the elastic tabs 78, this prevents the elastic tabs 78 from separating under the effect of the pressure exerted by the injection spring 24 and therefore makes it possible to retain the distal shoulder 30 of the injection syringe 12 (cf. FIG. 28).

It will be understood that as the elastic tabs 78 of the end section 16 ensure axial retention (with respect to X) of the injection syringe 12 in the distal direction of the automatic injection device 10, the pressure exerted by the injection spring 24 on the plunger rod 19 during injection produces pressure on the distal end of the injection syringe 12 but does not produce pressure at the level of the flange 38 of the injection syringe 12.

The steps of assembling the automatic injection device 10 will now be described.

Overall, assembly of the automatic injection device 10 comprises four main steps:
a step of assembling a distal subassembly comprising the syringe support 14,
a step of insertion of the injection syringe 12 into the syringe support 14 from its proximal end 14P,
a step of insertion of the positioning rod 50 preassembled with the plunger rod 19 onto the proximal end 14P of the syringe support 14, with one preferably being positioned axially with respect to the other by means of the axial positioning means 94,
a step of assembling a proximal subassembly, brought back onto the plunger rod 19, with the distal subassembly.

One first assembles a first subassembly, referred to as the distal subassembly. A first step for this comprises the interlocking of the end section 16 with the withdrawal member 18 by snap-locking of the lugs 74 into the recesses 84. The hooking tabs 66 of the withdrawal member 18 slide in the axial rails 86 of the end section 16 until the lugs 74 snap-lock into the recesses 84. The step is followed by a step of snap-locking of the syringe support 14 to the end section 16 by means of interlocking of the radial projections 52 (by means of the ramps 55) of the syringe support 14 with the proximal windows 80 of the end section 16. The protective springs 15a, 15b are slipped onto the guide pins 56 before snap-locking of the syringe support 14 to the end section 16 and maintained in place by the snap-locking of the two pieces.

One further assembles a second subassembly referred to as the proximal subassembly. For this purpose, one assembles the position control member 20, the needle control member 21, the plunger control member 22, the retaining ring 23, the injection spring 24, and the outer housing 25. More particularly, one first assembles the injection spring 24 on the proximal end of the control member of the plunger 22 while keeping it compressed by cooperation of the retaining ring 23 with the retractable stop 27 carried by the plunger control member 22. One then places these elements in one of the two half shells of the position control member 20, while further adding the needle control member 21 in a coupling position with the plunger control member 22. One then fixes the second half shell of the position control member 20 onto the first half shell, and one then slips the outer housing 25 around these elements, completing the assembly by snap-locking of the distal end of the outer housing 25 and that of the position control member 20, with this snap-locking nevertheless allowing the possibility of axial sliding, allowing the position control member 20 to move back with respect to the outer housing 25.

One further assembles a third subassembly referred to as the intermediate subassembly. This subassembly comprises the plunger rod 19 equipped with the positioning rod 50.

The injection syringe 12, equipped with its protective cap 42 and the plunger 40, further forms an additional separate element.

The automatic injection device 10 is assembled by inserting into the distal subassembly of the injection syringe 12, with the latter being inserted into the syringe support 14 from its proximal end. At the time of this insertion, as the end section 16 and the syringe support 14 are in an axially spaced position, the end section 16 does not enclose the four holding tabs 53 of the syringe support 14, which are thus free to move radially away during the passage of the protective cap 42 of the injection syringe 12. The elastic tabs 78 of the end section 16 are, by cooperation with the protective cap 42, maintained in their moved-away position, thus allowing the insertion in the distal direction, from the proximal end of the syringe support 14, of the injection syringe 12 equipped with the protective cap 42. Once the injection syringe 12 and the distal subassembly have been assembled, the intermediate subassembly is moved back into the proximal end of the syringe body 26 by cooperation of the positioning rod 50 with the axial notches 48 of the syringe support 14. One then moves the proximal subassembly back onto the plunger rod 19, in particular by inserting the blocking pin 107 of the needle control member 21 into the guiding rail 49 of the syringe support 14, and everything is then locked by rotation of the withdrawal member 18 with respect to the outer housing 25, producing in particular a rotation of the blocking pin 107 so that the external shoulder of the syringe support 14 is blocked between the blocking pin 107 and the guide rib 108.

It will be understood that this assembly is particularly simple to implement without requiring complex equipment.

The invention is not limited to the embodiments presented, and other embodiments will be clearly obvious to the person skilled in the art. Among the many possible variants, it will be noted that it is possible in particular to move the plunger rod 19 back onto the plunger 40 by screwing. In this case, the positioning rod 50 is assembled in a withdrawn position on the plunger rod 19, the plunger rod 19 is then screwed into place, and the positioning rod 50 is then slid until it wedges against the syringe support 14 and activates the axial positioning means.

1. An automatic injection device for liquid products having a distal end and an opposite proximal end, comprising:
an injection spring that is deformable between a compressed state prior to use of the automatic injection device and a relaxed state after injection,
a plunger control member configured to apply a force to a plunger under the action of the injection spring to permit movement of the plunger in the distal direction of the automatic injection device, the plunger control member carrying a stop that is retractable between an active retaining configuration of the injection spring in the compressed state and a retracted configuration in which it releases the injection spring,
a locking member of the retractable stop in its active configuration, this locking member being capable of assuming a locking position in which it exerts pressure on the retractable stop to prevent it from assuming its retracted configuration and an unlocking position in which the retractable stop is released to assume its retracted configuration.

2. The automatic injection device according to claim 1, wherein the locking member comprises a central locking pin provided in the proximal end of an outer housing of the automatic injection device.

3. The automatic injection device according to claim 1, wherein the proximal end of the outer housing comprises a radial surface recessed in the vicinity of the central locking pin so as to be crossed by an elastic tab carrying the retractable stop at least when the locking member is in the locking position.

4. The automatic injection device according to claim 3, wherein the central locking pin comprises two sections distributed axially and of different diameters, namely a locking section and an unlocking section, the locking section having a radial dimension that is greater than a radial dimension of the unlocking section when viewing an axial section through the retractable stop, such that the locking section exerts a radial pressure on the retractable stop when the locking member is in the locking position, and the unlocking section creates a space for disengaging the retractable stop when the locking member is in the unlocking position.

5. The automatic injection device according to claim 1, wherein the locking member carries a ramp configured to cooperate by ramp effect with an elastic tab carrying the retractable stop in order to shift it from the active configuration to the retracted configuration.

6. The automatic injection device according to claim 1, wherein the passage from the locking position to the unlocking position of the locking member is triggered by application of the distal end of the automatic injection device to the skin of a patient.

7. The automatic injection device according to claim 1, wherein the retractable stop is radially displaced when it is in its active configuration, in particular such that it assumes a radially projecting position, and the retractable stop is radially retracted when it is in its retracted configuration, in particular such that it assumes a radially retracted position.

8. The automatic injection device according to claim 1, wherein the retractable stop is carried by an elastic tab extending axially towards the proximal end of the automatic injection device, the elastic tab being deformable between:
a radially displaced rest configuration giving the retractable stop its active configuration, and
a radially retracted configuration giving the retractable stop its retracted configuration.

9. The automatic injection device according to claim 1, wherein the retractable stop in its active configuration rests against a complementary surface carried by a spring retaining ring, this retaining ring being attached to a position control member comprising a cam track for controlling the movement of elements of the automatic injection device under the effect of the injection spring.

10. The automatic injection device according to claim 1, wherein the plunger control member comprises two diametrically opposed semi-annular elastic tabs, each carrying a stop that is retractable, each semi-annular elastic tab having a longitudinal window allowing deformation in order to facilitate the assembly of the injection spring and the retaining ring of the spring around the two semi-annular elastic tabs.

11. The automatic injection device according to claim 2, wherein the central locking pin comprises two sections distributed axially and of different diameters, namely a locking section and an unlocking section, the locking section having a radial dimension that is greater than a radial dimension of the unlocking section when viewing an axial section through the retractable stop, such that the locking section exerts a radial pressure on the retractable stop when the locking member is in the locking position, and the unlocking section creates a space for disengaging the retractable stop when the locking member is in the unlocking position.
